(19) United States
(12) Patent Application Publication
(10) Pub. No.: US 2022/0226529 A1
(43) Pub. Date: Jul. 21, 2022

(54) SYSTEM FOR DETERMINING ACHIEVEMENT DEGREE OF STERILIZATION PROCESS, DETERMINATION APPARATUS, MEASUREMENT APPARATUS, AND COMPUTER-READABLE STORAGE MEDIUM

(71) Applicant: CANON KABUSHIKI KAISHA, Tokyo (JP)

(72) Inventor: Toshifumi KItamura, Shizuoka (JP)

(21) Appl. No.: 17/607,099

(22) PCT Filed: Jun. 17, 2020
(86) PCT No.: PCT/JP2020/023766

§ 371 Date: Oct. 28, 2021

(30) Foreign Application Priority Data
Jun. 24, 2019 (JP) 2019-116632

(51) Int. Cl. A61L 2/28; G01N 31/22

(52) U.S. Cl. CPC A61L 2/28; A61L 2202/14; G01N 31/226

(57) ABSTRACT

A determination system includes: type determination means for acquiring determination information for determining a type of an indicator that has a discoloration area, a color of which changes according to an achievement degree of a sterilization process, and determining the type of the indicator; measurement means for measuring color information regarding the discoloration area; and achievement degree determination means for determining the achievement degree of the sterilization process based on the color information regarding the discoloration area measured by the measurement means, according to a determination criterion corresponding to the type of the indicator determined by the type determination means.

### TECHNICAL FIELD

The present invention relates to a technique for determining an achievement degree of a sterilization process, using an indicator.

### BACKGROUND ART

In hospitals, objects that are to be sterilized (sterilization targets), such as medical tools, are subjected to a sterilization process. To determine the achievement degree of the sterilization process for a sterilization target, so-called sterilization indicators, such as a chemical indicator (hereinafter referred to as a CI) are used. A CI has a discoloration area that discolors according to the achievement degree of conditions for sterilization process that involves a sterilizer (steam, hydrogen peroxide, etc.). A common method for checking the achievement degree of a sterilization process is to visually check changes in the color of the discoloration area of a CI. Japanese Patent Laid-Open No. 2003-325646 discloses a measurement apparatus that measures the achievement degree of a sterilization process by optically reading the color of the discoloration area of a CI.

Here, a different CI is used depending on the sterilization method employed in the sterilization apparatus. Note that different sterilization methods employ different sterilizers or different sterilization processes, for example. In addition, CIs vary in the position of the discoloration area, and the color before and after discoloration. In hospitals or the like, a plurality of sterilization apparatuses that employ different sterilization methods are used, and accordingly a plurality of types of CIs are used. CIs vary depending on the type thereof, in the way in which the color of the discoloration area changes, and the position of the discoloration area. Therefore, with the measurement apparatus disclosed in Japanese Patent Laid-Open No. 2003-325646, it is necessary to change the measurement settings of the measurement apparatus and determination conditions for determining the achievement degree of a sterilization process according to the type of the CI that is to be measured. Thus, the measurement apparatus disclosed in the Japanese Patent Laid-Open No. 2003-325646 increases a burden on a user, and degrades workability.

### SUMMARY OF INVENTION

According to an aspect of the present invention, a determination system includes: type determination means for acquiring determination information for determining a type of an indicator that has a discoloration area, a color of which changes according to an achievement degree of a sterilization process, and determining the type of the indicator; measurement means for measuring color information regarding the discoloration area; and achievement degree determination means for determining the achievement degree of the sterilization process based on the color information regarding the discoloration area measured by the measurement means, according to a determination criterion corresponding to the type of the indicator determined by the type determination means.

Further features of the present invention will become apparent from the following description of exemplary embodiments with reference to the attached drawings.

### BRIEF DESCRIPTION OF DRAWINGS

FIG. 1 is a configuration diagram of a determination system according to an embodiment.

FIGS. 2A to 2C are configuration diagrams of a spectrometer.

FIG. 3 is a functional block diagram of a determination system.

FIG. 4 is a diagram showing a CI.

FIGS. 5A and 5B are diagrams showing examples of different types of CIs.

FIGS. 6A and 6B are diagrams showing examples of the profiles of the CIs.

FIG. 7 is a flowchart for a measurement process that is carried out by a measurement apparatus according to an embodiment.

FIG. 8 is a diagram showing an example of a screen that is displayed by a determination apparatus.

FIG. 9 is a configuration diagram of a determination system according to an embodiment.

FIG. 10 is a configuration diagram of a determination system according to an embodiment.

FIG. 11 is a flowchart for a measurement process that is carried out by a measurement apparatus according to an embodiment.

FIG. 12 is a flowchart for a determination process that is carried out by a determination apparatus according to an embodiment.

FIG. 13 is a flowchart for a measurement process that is carried out by a measurement apparatus according to an embodiment.

### DESCRIPTION OF EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

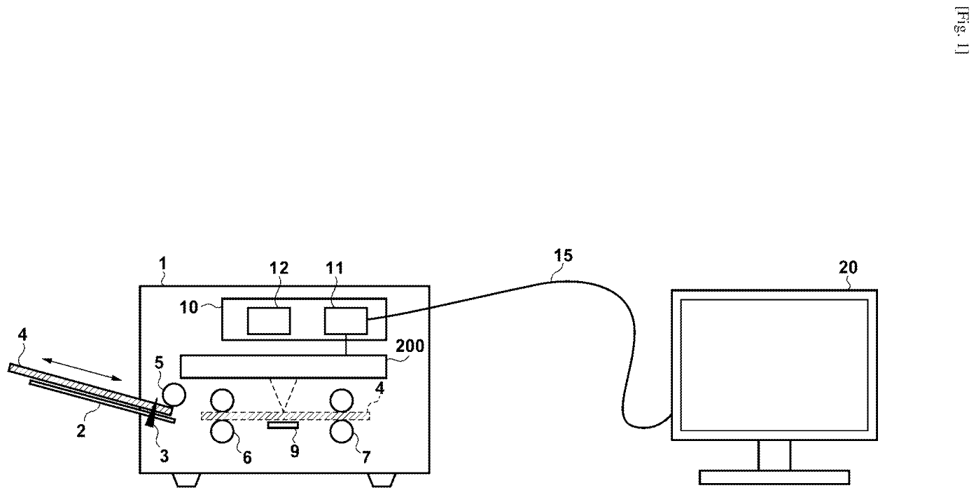

FIG. 1 is a configuration diagram of a determination system for determining the achievement degree of a sterilization process according to the present embodiment. The determination system includes a measurement apparatus 1 for a CI 4, and a determination apparatus 20. The determination apparatus 20 may be constituted by a personal computer (hereinafter referred to as a PC), a tablet, or the like. The measurement apparatus 1 and the determination apparatus 20 are connected to each other via a communication line 15. Although the measurement apparatus 1 and the determination apparatus 20 communicate with each other via the communication line 15 in the present embodiment, the measurement apparatus 1 and the determination apparatus 20 may be configured to wirelessly communicate with each other. Furthermore, the measurement apparatus 1 and the determination apparatus 20 may be configured to communicate with each other via a network such as a wired or a wireless LAN.

A sensor 3 of the measurement apparatus 1 detects whether or not the CI 4 is placed on a tray 2. A roller 5 conveys the CI 4 to the inside of the measurement apparatus 1. The CI 4 conveyed to the inside of the measurement apparatus 1 is conveyed to a measurement area of a spectrometer 200 by an upstream roller 6 and a downstream roller 7. The spectrometer 200 measures the color of the surface of the CI 4, and outputs the result of measurement to a control unit 10. The control unit 10 includes a CPU 11 and a nonvolatile memory 12. Note that a white reference plate 9 for color correction of the spectrometer 200 is provided between the upstream roller 6 and the downstream roller 7. When the CI 4 is not located between the spectrometer 200 and the white reference plate 9, the spectrometer 200 can measure the color of the surface of the white reference plate 9. After measurement by the spectrometer 200 is complete, the CI 4 is ejected onto the tray 2 by the upstream roller 6 and the downstream roller 7. The control unit 10 transmits the result of measurement of the CI 4 to the determination apparatus 20. Based on the result of measurement, the determination apparatus 20 determines the achievement degree of the sterilization process, and displays the result of determination and other reference information on a display unit such as a display.

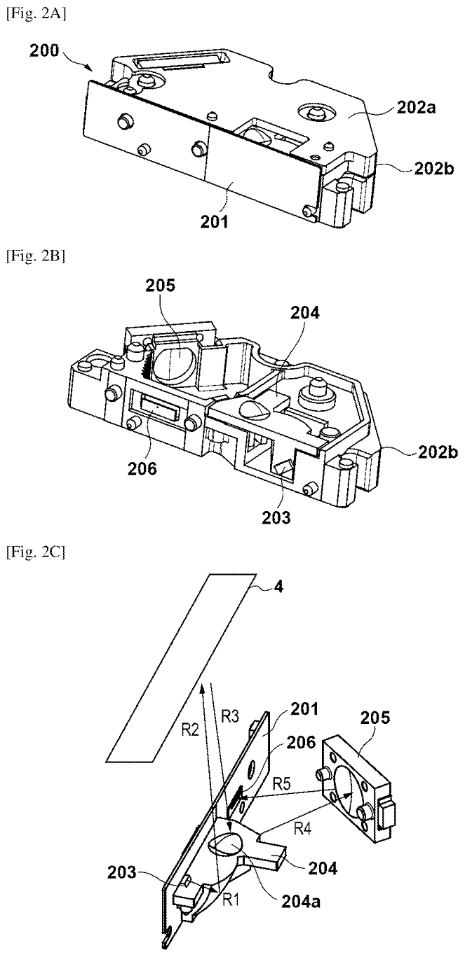

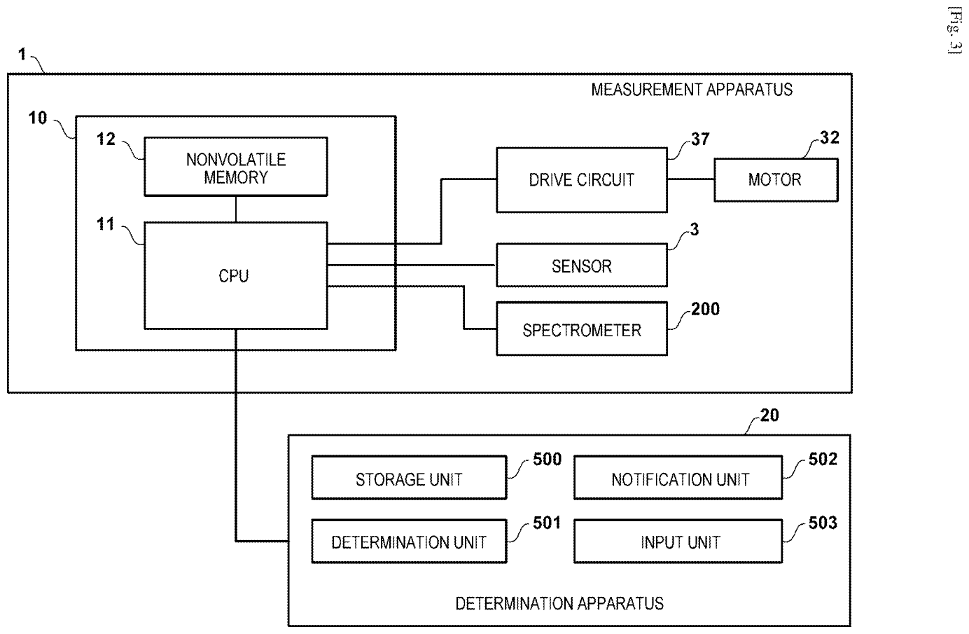

FIGS. 2A to 2C are configuration diagrams of the spectrometer 200. FIG. 2A shows an external appearance of the spectrometer 200, and FIG. 2B shows a state in which a substrate 201 and a cover member 202a, which are shown in FIG. 2A, have been removed from a housing 202b of the spectrometer 200. The substrate 201 is provided with, for example, a white LED 203, a line sensor 206, and a circuit for amplifying an output signal from the line sensor 206 and converting the signal into a digital signal. Note that FIG. 2B shows that the white LED 203 and the line sensor 206 are located at positions at which the white LED 203 and the line sensor 206 are located when the substrate 201 is attached to the housing 202b as shown in FIG. 2A. A light guide 204 is a light guide member into which an illumination portion that guides light emitted by the white LED 203, to the measurement target, and a light collecting portion that collects and guides light from the measurement target, are integrated. Light emitted by the white LED 203 illuminates the measurement target, i.e. the CI 4, via the light guide 204. Reflection light from the measurement target is collected and guided by the light collecting portion of the light guide 204 and illuminates a diffraction grating 205. The diffraction grating 205 separates the reflection light according to wavelengths. The line sensor 206 includes a plurality of light receiving elements, and each light receiving element receives light in a predetermined wavelength range separated by the diffraction grating 205.

FIG. 2C illustrates paths of light emitted from the white LED 203 and entering the line sensor 206. A light ray R1 emitted by the white LED 203 is reflected off a curved surface of the light guide 204, and illuminates the CI 4 as an illumination light ray R2. A reflection light ray R3 from the CI 4 enters a light entrance portion 204a of the light guide 204. The reflection light ray that has entered the light entrance portion 204a is collected and guided by the light guide 204, and illuminates the diffraction grating 205 as a reflection light ray R4. A reflection light ray R5 that has been separated by the diffraction grating 205 enters the line sensor 206.

FIG. 3 is a functional block diagram of the measurement apparatus 1 and the determination apparatus 20. The CPU 11 controls a drive circuit 37 to control the rotation of one or more motors 32. The one or more motors 32 rotate the roller 5, the upstream roller 6, and the downstream roller 7. The CPU 11 acquires the result of detection performed by the sensor 3. The CPU 11 also controls measurement of the CI 4 performed by the spectrometer 200. The CPU 11 also receives a signal indicating the amount of received light, from each light receiving element of the line sensor 206 of the spectrometer 200, and obtains color information regarding the surface of the CI 4. The color information to be obtained by the CI 4 may be color information in any color space. The nonvolatile memory 12 holds programs that are to be executed by the CPU 11, and various kinds of information that are to be used by the CPU 11 in order to measure the CI 4. The CPU 11 measures the CI 4 by executing a program held by the nonvolatile memory 12, and using various kinds of information held by the nonvolatile memory 12.

The determination apparatus 20 includes a determination unit 501 that determines the achievement degree of a sterilization process based on the result of measurement of the CI 4 performed by the measurement apparatus 1. A notification unit 502 of the determination apparatus 20 notifies the user of the result of determination by the determination unit 501 and reference information. Note that, in the present embodiment, the notification unit 502 displays the result of determination, etc. on a display (a display unit) (not shown) of the determination apparatus 20. The determination unit 501 and the notification unit 502 can be realized by, for example, causing one or more processors of the PC to execute an appropriate program. Note that such a program is stored in a storage unit 500 such as a hard disk drive. The storage unit 500 also holds various kinds of information that are to be used by the determination unit 501. The user can input various kinds of information to the determination apparatus 20 by using an input unit 503.

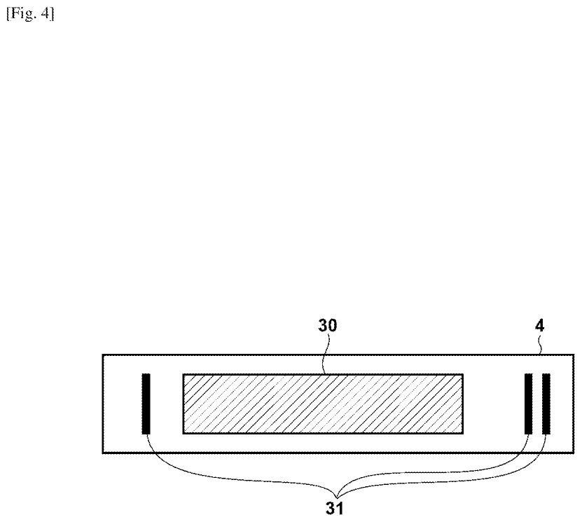

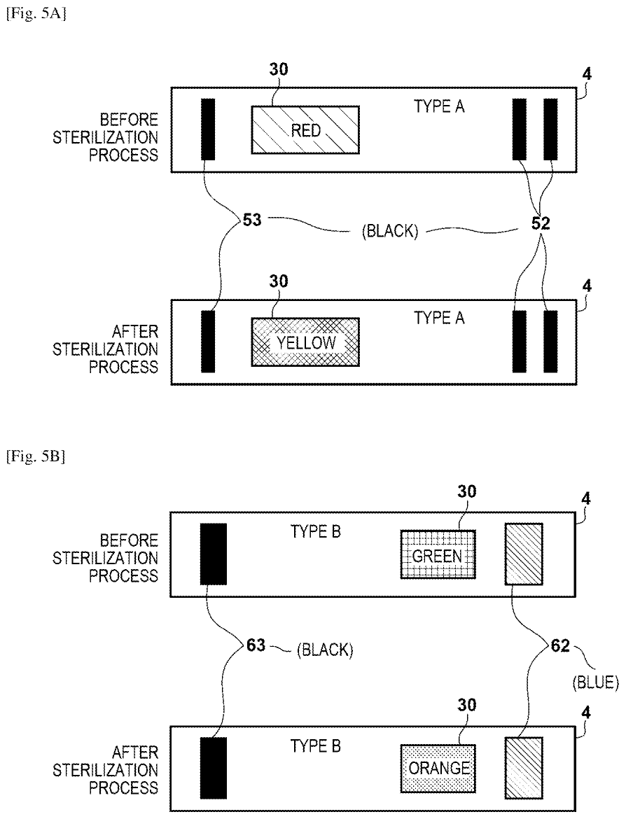

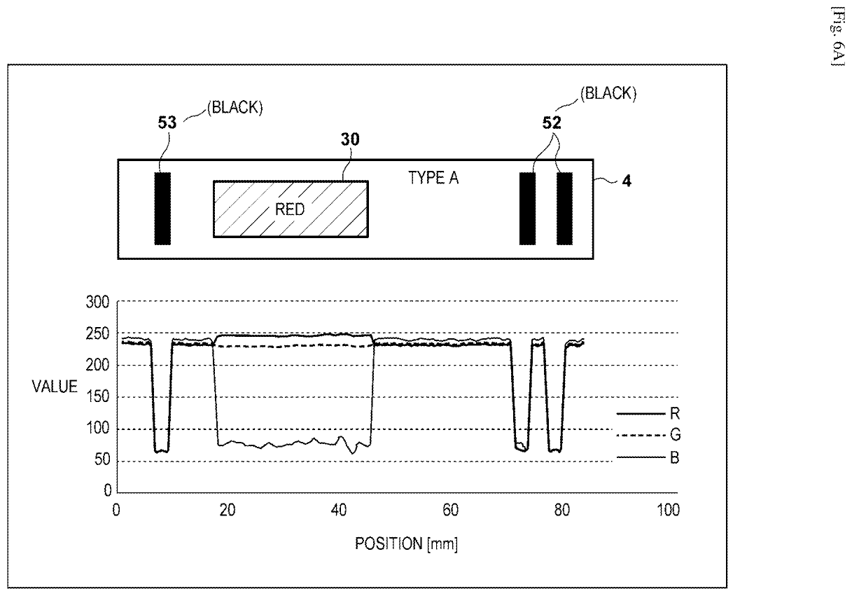

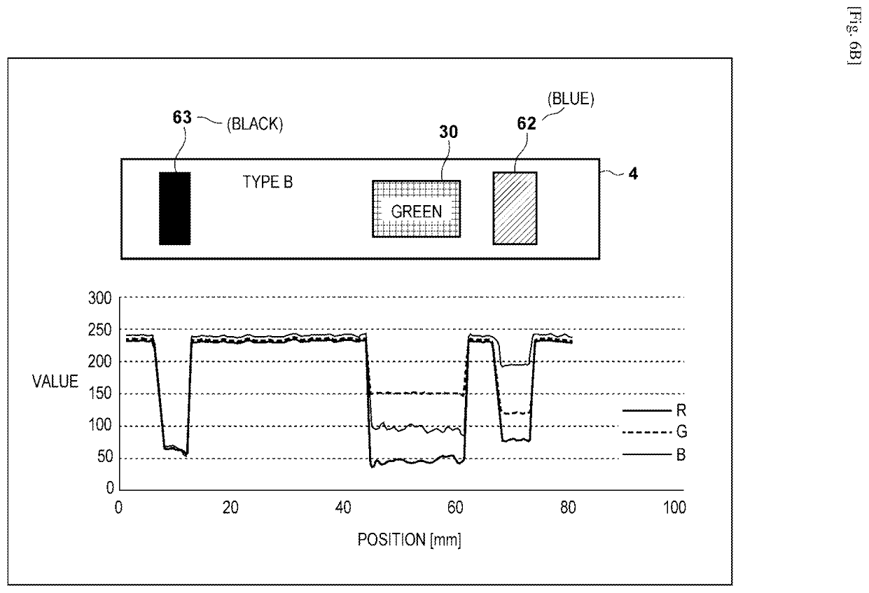

FIG. 4 shows an example of the CI 4. The CI 4 is a sheet of test paper that has a discoloration area 30, which has been chemically processed, on the surface thereof. The color of the discoloration area 30 changes according to the achievement degree of the sterilization process. Identification patterns 31 are used to identify the type of the CI 4. In the present embodiment, the identification patterns 31 are printed on the surface on the same side as the discoloration area 30 of the CI 4, at positions different from the position of the discoloration area 30. Note that the measurement apparatus 1 is configured to convey the CI 4 in the lengthwise direction of the CI 4. FIGS. 5A and 5B show other examples of the CI 4. The CI 4 shown in FIG. 5A is hereinafter referred to as a type A, and the CI 4 shown in FIG. 5B is hereinafter referred to as a type B. The color of the discoloration area 30 of the type A is red before the sterilization process, and changes to yellow as a result of the sterilization process. In contrast, the color of the discoloration area 30 of the type B is green before the sterilization process, and changes to orange as a result of the sterilization process. Furthermore, the position and the size of the discoloration area 30 are different in the type A and the type B. Note that the identification patterns 31 of the type A are constituted by one black line 53 near the short side that is closer to the discoloration area 30, and two black lines 52 near the other short side. In contrast, the identification patterns 31 of the type B are constituted by a blue square 62 near the short side that is closer to the discoloration area 30, and a black square 63 near the other short side.

FIGS. 6A and 6B respectively show the profiles of the type A and the type B, regarding the color of the surface of the CI 4 before the sterilization process. Note that the horizontal axis indicates a position on the CI 4 and the vertical axis indicates color information obtained through measurement. In these examples, color information is a color value in an RGB color space. In the present embodiment, a profile regarding the color of the CI (hereinafter simply referred to as a profile) is information regarding the color of the CI 4 in a predetermined range in a predetermined direction. Note that the predetermined direction is a direction that connects the identification patterns 31 and the discoloration area 30, and is specifically a direction that is parallel with the long sides of the CI 4. In FIGS. 6A and 6B, the predetermined range is the entire range of the CI 4 in the lengthwise direction thereof. Although the color information in the present embodiment is a color value in an RGB color space, a color value in any color space may be used. Alternatively, the amount of received light of each wavelength, acquired by the spectrometer 200, may be used as a color value. As shown in FIGS. 6A and 6B, the profile of the CI 4 varies depending on the type thereof. Also, as shown in FIGS. 6A and 6B, when the profile is not bilaterally symmetrical, it is possible to determine the orientation of the CI 4, i.e. which short side of the two short sides is the leading end of the CI 4 that is being conveyed, based on the profile. Although the FIGS. 6A and 6B show the profiles before the sterilization process is complete, the same applies to the profiles after the sterilization process is complete.

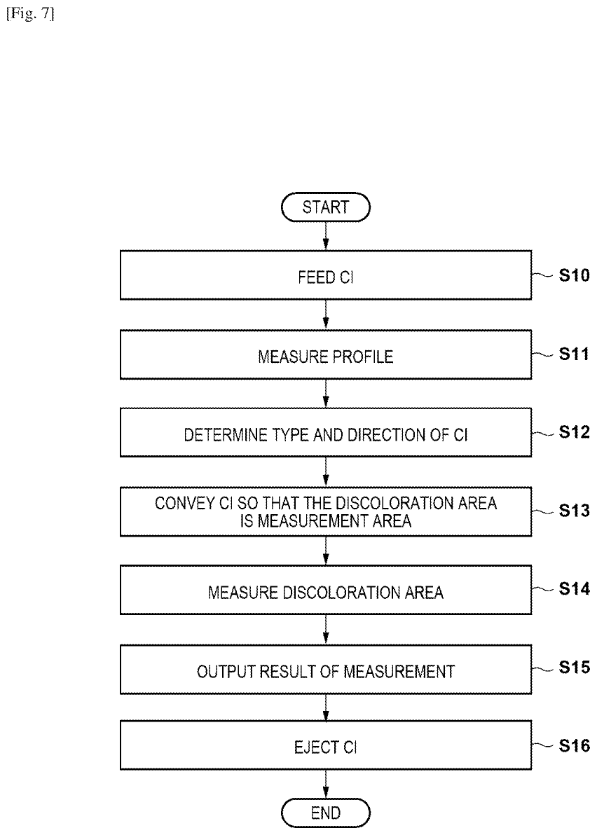

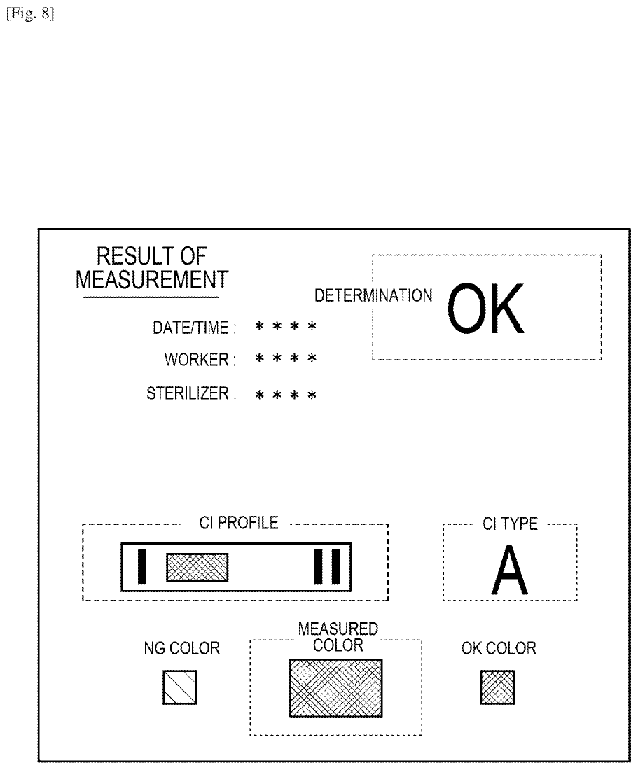

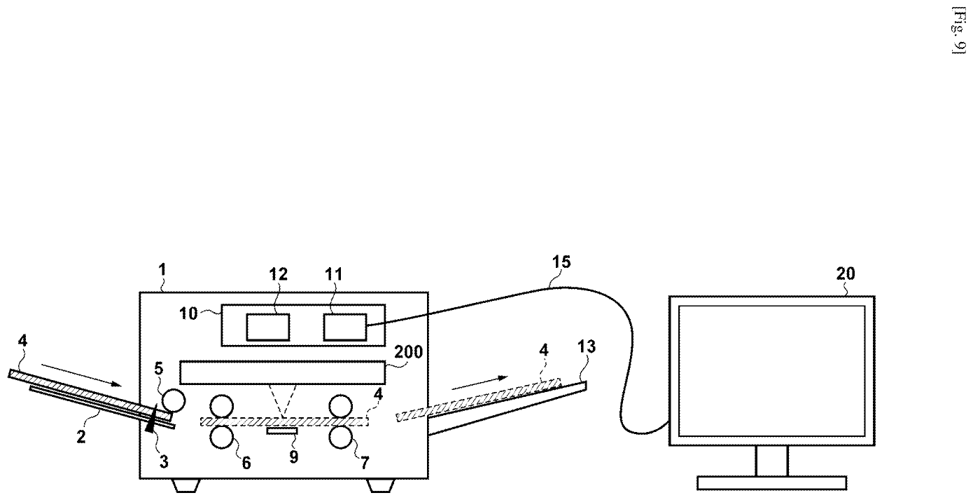

FIG. 7 is a flowchart for a measurement process that is carried out by the control unit 10 of the measurement apparatus 1 to measure the CI 4. The control unit 10 starts the process shown in FIG. 7 upon receiving a measurement instruction from the determination apparatus 20. Upon receiving a measurement instruction from the determination apparatus 20, the control unit 10 monitors the state of detection by the sensor 3 of the CI 4 in step S10. Upon the sensor 3 detecting the CI 4, the control unit 10 conveys the CI 4 on the tray 2 to the inside of the measurement apparatus 1. In step S11, the control unit 10 measures the profile of the CI 4 from when the leading end of the CI 4 reaches the measurement area of the spectrometer 200 until when the trailing end of the CI 4 has passed through the measurement area.

In step S12, the control unit 10 determines the type and the orientation of the CI 4 based on the measured profile of the CI 4. In the present embodiment, the nonvolatile memory 12 stores profiles of a plurality of types of CIs 4 that are to be used, as determination information for determining the type of each CI 4. Note that the profiles of the respective types of CIs 4 indicated by determination information are profiles after a standard sterilization process. Although the profile of the CI 4 to be measured varies depending on which of the two short sides of the CI 4 is the leading end of the CI 4 when conveyed, determination information is acquired when the CI 4 is conveyed such that the short side that serves as a reference is the leading end. The control unit 10 selects a profile that is most similar to the profile measured in step S11, from among profiles that are indicated by determination information. Note that a selection criterion has been determined in advance, and the most similar profile is determined according to the selection criterion. For example, the selection criterion may be as follows: the measured profile of an area that includes the identification patterns 31 at the leading end and the trailing end of the CI 4 in the conveyance direction is compared with the profiles indicated by the determination information, and the profile with the smallest difference is selected. Alternatively, the selection criterion may be as follows: the entire measured profile is compared with the profiles indicated by the determination information, and the profile with the smallest difference is selected. As described above, the profile of the CI 4 to be measured varies depending on which of the two short sides of the CI 4 is the leading end of the CI 4 when conveyed. Therefore, the control unit 10 compares the reverse of the measured profile of the CI 4, in addition to the measured profile of the CI 4 per se, with the profiles indicated by the determination information. The control unit 10 determines the type corresponding to the selected profile, as the type of the CI 4. In this way, the control unit 10 functions as a type determination unit that determines the type of the CI 4.

The determination information also includes information regarding the position (the range) of the discoloration area 30 for each type of CI 4. For example, the position of the discoloration area 30 is indicated as a distance from the reference short side of the CI 4. The control unit 10 can determine whether the reference short side of the CI 4 that is being conveyed is on the leading end side or the trailing end side based on whether or not the measured profile is the reverse of the profile selected from the determination information. Therefore, the control unit 10 can determine the position of the discoloration area 30 of the CI 4 based on information indicating the position (the range) of the discoloration area 30 and the result of determination regarding whether the reference short side of the CI 4 is on the leading end side or the trailing end side. In this way, the control unit 10 also functions as a position determination unit that determines the position of the discoloration area 30 of the CI 4. The determination information also serves as information used to determine the position of the discoloration area 30 of the CI 4. In step S13, based on the result of determination regarding the position of the discoloration area 30, the control unit 10 adjusts the position of the CI 4 such that the discoloration area is within the measurement area of the spectrometer 200, using the upstream roller 6 and the downstream roller 7. Thereafter, in step S14, the control unit 10 measures the color of the discoloration area 30 of the CI 4, using the spectrometer 200. In step S15, upon the measurement being complete, the control unit 10 outputs the result of measurement to the determination apparatus 20, and ejects the CI 4 from the measurement apparatus 1 to the tray 2 in step S16.

Note that the result of measurement transmitted to the determination apparatus 20 in step S15 includes the result of measurement of the discoloration area 30 and information indicating the determined type of the CI 4. Upon receiving the result of measurement from the measurement apparatus 1, the determination unit 501 (the achievement degree determination unit) of the determination apparatus 20 determines the achievement degree of the sterilization process based on the result of measurement of the discoloration area 30. Specifically, for each type of CI 4, a determination criterion indicating the relationship between the color value of the discoloration area 30 and the achievement degree of the sterilization process is stored in advance in the storage unit 500 of the determination apparatus 20, as determination criterion information. Based on the received type of the CI 4, the determination unit 501 of the determination apparatus 20 selects the determination criterion of that type from the determination criterion information. Thereafter, the determination unit 501 determines the achievement degree of the sterilization process based on the received color value of the discoloration area 30 and the selected determination criterion. The notification unit 502 of the determination apparatus 20 notifies the user of the result of determination. Furthermore, the notification unit 502 may be configured to display the measured color of the discoloration area 30 and the determined type of the CI 4 as reference information. FIG. 8 shows an example of a screen that is to be displayed for the user. In FIG. 8, the determined type of CI 4 (CI TYPE in FIG. 8) and the measured color of the discoloration area (MEASURED COLOR in FIG. 8) are displayed in addition to the result of determination (OK in FIG. 8). Furthermore, in FIG. 8, a representative color indicating that the sterilization process for the determined type of CI 4 is OK (OK COLOR) and a representative color indicating that the sterilization process is NG (unacceptable) (NG COLOR) are also displayed. Note that the measurement apparatus 1 may be configured to also transmit the measured profile of the CI 4 to the determination apparatus 20 in step S15 as a result of measurement. In this example, the measurement apparatus 1 also transmits the profile to the determination apparatus 20, and thus the determination apparatus 20 displays the measured profile (CI PROFILE in FIG. 8) as a color.

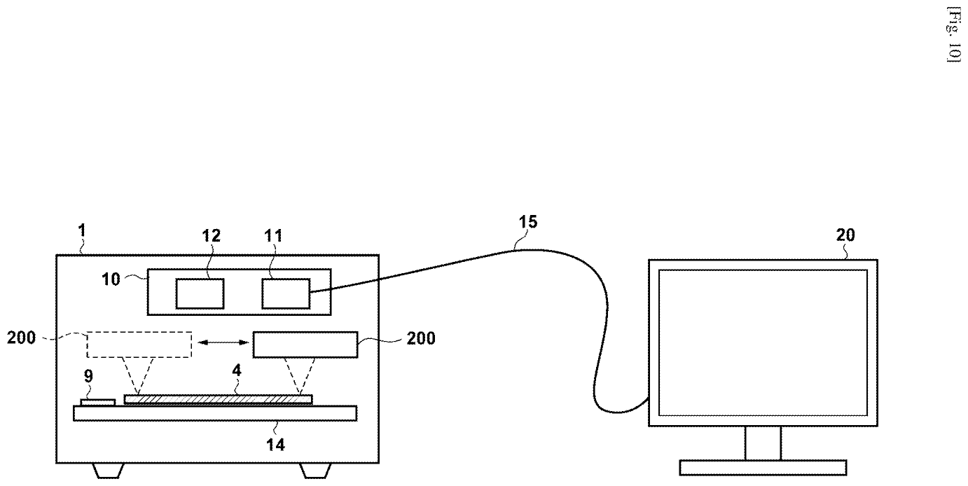

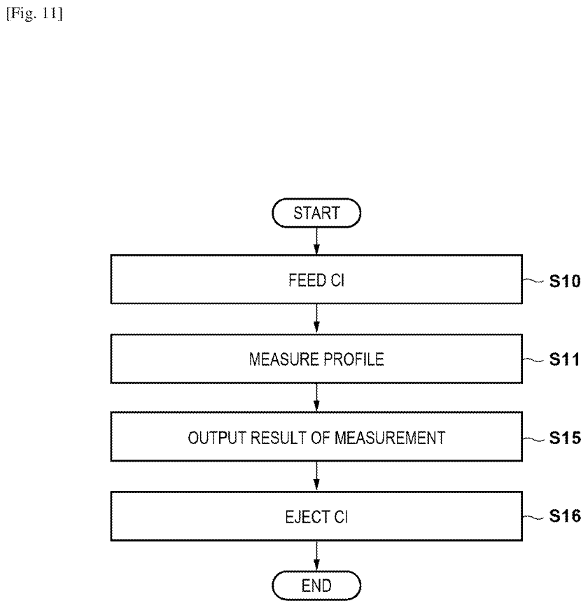

In the present embodiment, the measurement apparatus 1 takes in the CI 4 from the tray 2 and performs measurement, and ejects the CI 4 to the tray 2 again after the measurement. However, as shown in FIG. 9, it is possible to employ a configuration in which the measurement apparatus 1 takes in the CI 4 from the tray 2 and performs measurement, and thereafter ejects the CI 4 onto a tray 13 that is provided on the opposite side to the tray 2. Also, in the measurement apparatus 1 shown in FIGS. 1 and 9, the position of the spectrometer 200 is fixed, and accordingly the measurement area of the spectrometer 200 is fixed. However, as shown in FIG. 10, it is possible to employ a configuration in which the spectrometer 200 can be moved and the measurement area of the spectrometer 200 can be moved accordingly. In FIG. 10, the user places the CI 4 on a measurement platform 14. Thereafter, the spectrometer 200 is moved instead of the CI 4, and thus the profile and the discoloration area 30 of the CI are measured. With the configuration shown in FIG. 10, it is unnecessary to provide a conveyance system for conveying the CI 4, or a tray, and thus the measurement apparatus 1 can be downsized.

Note that, in the present embodiment, the profile of the CI 4 is measured while the relative positional relationship between the spectrometer 200 and the CI 4 is changed by the spectrometer 200. However, the present invention is not limited to a configuration in which color information regarding the surface of the CI 4 is acquired by the spectrometer 200. For example, it is possible to employ a configuration for acquiring color information regarding the surface of the CI 4, using an RGB sensor. Alternatively, it is also possible to employ a configuration for measuring the type of the CI 4 and the color of the discoloration area 30 from image data obtained by capturing an image of the CI 4 using an imaging device such as a CIS (contact image sensor). Note that, when an imaging device is used, an image is captured in a state where the conveyance of the CI 4 has been stopped.

Note that it is possible to employ a configuration with which the processes carried out by the determination apparatus 20 in the present embodiment are carried out by an application on a cloud, and the determination apparatus 20 only acquires information that the user is to be notified of by the notification unit 502, from the cloud. Also, although the entire range of the profile of the CI 4 in the conveyance direction of the CI 4 is measured, it is possible to employ a configuration for measuring a predetermined range in the conveyance direction of the CI 4 if there will be no influence on the determination of the type and the orientation of the CI. For example, a range that includes the identification patterns 31 and the discoloration area 30 may be defined as the predetermined range. Furthermore, the predetermined range may be a range that only includes the identification patterns 31, a range that only includes the discoloration area 30, or a range that includes some or all of the identification patterns 31 and at least a portion of the discoloration area 30. Also, although the identification patterns 31 of the CI 4 shown in FIGS. 4, 5A, and 5B are provided near the two short sides of the CI 4, it is possible to employ a configuration in which the identification patterns 31 are provided near only one of the two short sides.

In the present embodiment, the notification unit 502 displays the result of determination and reference information on a display, which is a display unit for the determination apparatus. However, it is possible to employ a configuration in which the notification unit 502 stores the result of determination and reference information in the storage unit 500, or output them to a printer. Also, when the determination apparatus 20 is connected to a network, the notification unit 502 may be configured to transmit the result of determination and reference information to another computer.

As described above, according to the present embodiment, the measurement apparatus 1 determines the type and the orientation of the measurement target CI 4, and thus the measurement apparatus 1 can correctly measure the discoloration area 30 of the CI 4, the position of which may vary depending on the type thereof, according to the determined type and the orientation of the CI 4. Also, by notifying the determination apparatus 20 of the determined type, the determination apparatus 20 can determine the achievement degree of the sterilization process according to a determination criterion that may vary depending on the type of the CI. With such a configuration, even when a plurality of types of CIs 4 are used in the sterilization process, it is possible to improve workability regarding the determination of the achievement degree of the sterilization process.

### Second Embodiment

The following describes a second embodiment, mainly regarding differences from the first embodiment. In the first embodiment, after determining the type and the orientation of the CI 4, the measurement apparatus 1 adjusts the position of the CI 4 such that the discoloration area 30 of the CI 4 will be located within the measurement area of the spectrometer 200, and thereafter measures the color of the discoloration area 30. In the present embodiment, such an adjustment of the position of the CI 4 is not performed.

FIG. 11 is a flowchart for a measurement process that is carried out by the control unit 10 to measure the CI 4 in the present embodiment. The difference from the first embodiment is that the processes in steps S12 to S14 shown in FIG. 7 are omitted. In step S15, the control unit 10 only outputs the measured profile of the CI 4 to the determination apparatus 20. Note that, in the present embodiment, the predetermined range of the CI 4 from which the measurement apparatus 1 acquires the profile includes at least a portion of the discoloration area 30.

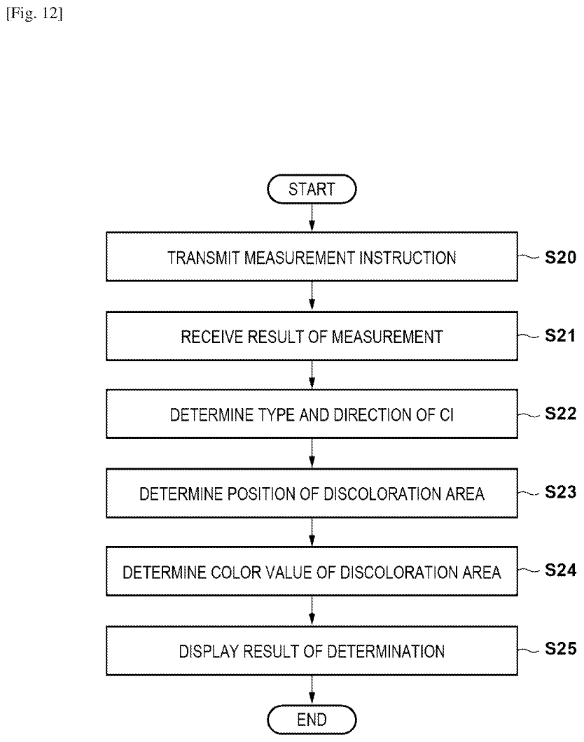

FIG. 12 is a flowchart for a determination process that is carried out by the determination apparatus 20 to determine the achievement degree of the sterilization process according to the present embodiment. Note that the determination unit 501 starts the process in FIG. 12 upon an instruction to measure the CI 4 being input thereto from the user via the input unit 503. In step S20, the determination unit 501 transmits a measurement instruction to the measurement apparatus 1. Thus, the measurement apparatus 1 carries out the process in FIG. 11. Therefore, the determination unit 501 receives the result of measurement, the profile of the CI 4, from the measurement apparatus 1 in step S21. In step S22, the determination unit 501 determines the type of the CI 4 and the direction of measurement of the CI 4 in the measurement apparatus 1, based on the profile of the CI 4. The determination of the type and the direction of measurement of the CI 4 are the same as those in the first embodiment. That is to say, in the present embodiment, the storage unit 500 stores determination information. In step S23, the determination unit 501 determines the position of the discoloration area 30 of the CI 4 according to the determined type and the orientation of the CI 4. In step S24, the determination unit 501 determines a portion of the profile corresponding to the discoloration area 30 in the profile of the CI 4 based on the determined position of the discoloration area 30, and thus determines the color value of the discoloration area 30. Thereafter, in step S25, the determination apparatus 20 displays the result of determination of the sterilization process, for example, for the user as in the first embodiment.

As described above, in the present embodiment, it is unnecessary to adjust the position of the CI 4 so that the discoloration area 30 of the CI 4 will be located within the measurement area of the spectrometer 200, and it is possible to reduce the time required for measurement. In particular, in the case of the measurement apparatus 1 shown in FIG. 9, it is unnecessary to reverse the conveyance direction of the CI 4, and it is possible to further reduce the time required to carry out measurement of the CI 4. Similarly, in the case of the measurement apparatus 1 shown in FIG. 10, it is unnecessary to reverse the moving direction of the spectrometer 200, and it is possible to further reduce the time required to carry out measurement of the CI 4. Also, it is possible to determine the type of the CI 4 based on the profile, and determine the achievement degree of the sterilization process according to the determination criterion corresponding to the determined type of the CI 4. Thus, it is possible to improve workability when determining the achievement degree even when a plurality of types of CIs 4 are used.

Note that, in the first embodiment, the measurement apparatus 1 measures the color information regarding the discoloration area 30 after measuring the profile of the CI 4. However, in the first embodiment, as in the present embodiment, the measurement apparatus 1 may be configured to determine a portion corresponding to the discoloration area 30 in the profile of the CI 4, and thus determine the color value of the discoloration area 30 and transmits it to the determination apparatus 20.

### Third Embodiment

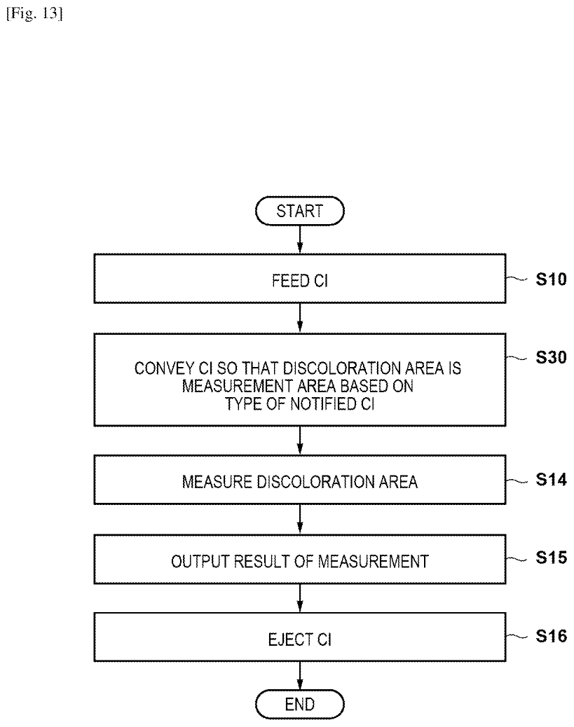

The following describes a third embodiment, mainly regarding differences from the first embodiment. In the first embodiment, the measurement apparatus 1 measures the profile of the CI 4, and thus determines the type and the orientation of the CI 4. In the present embodiment, the user inputs the type of the CI 4 to the determination apparatus 20 based on the identification patterns 31 of the CI 4, for example. For example, the notification unit 502 of the determination apparatus 20 displays a relationship between identification patterns and types for the user, and the user can determine the type of the CI 4 based on the content of display. Upon the user inputting the type of the CI 4 via the input unit 503, the notification unit 502 displays the orientation of the CI 4 when placed on the tray 2, on the display. Based on the content displayed on the display, the user places the CI 4 on the tray 2. FIG. 13 is a flowchart for a measurement process that is carried out by the control unit 10 to measure the CI 4 in the present embodiment. The difference from the first embodiment is that, after the CI 4 has been fed in step S10 in FIG. 7, the control unit 10, in step S30, conveys the CI 4 according to the type of the CI notified by the determination apparatus 20 so that the discoloration area 30 will be located within the measurement area of the spectrometer 200. The subsequent processes in steps S14 to S16 are the same as in the first embodiment. However, in step S15, the control unit 10 outputs the result of measurement of the discoloration area 30 to the determination apparatus 20. Note that, regardless of the type of the CI 4 to be used, if the discoloration area is located within a predetermined area of the CI 4, i.e. within an area that includes the center position in the conveyance direction of the CI 4, for example, the orientation of the CI 4 when placed on the tray 2 may be any orientation. Therefore, it is unnecessary to notify the user of the orientation of the CI 4.

As described above, according to the present embodiment, the user inputs the type of the CI 4 to the determination apparatus 20, and thus the measurement apparatus 1 can measure the discoloration area 30 of the CI 4. Therefore, it is possible to reduce the time required to carry out measurement. Also, the determination apparatus 20 can determine the achievement degree of the sterilization process based on the determination criterion corresponding to the type of the CI 4 input by the user, and it is possible to improve workability.

### Fourth Embodiment

The following describes a fourth embodiment, regarding differences from the first embodiment and the third embodiment. In the present embodiment, a wireless tag such as an RFID is attached to the CI 4 instead of the identification patterns 31, or in addition to the identification patterns 31. The wireless tag stores determination information that indicates the type of the CI 4. In the third embodiment, the user determines the type of the CI 4 based on the identification patterns 31, for example, and inputs the type to the determination apparatus 20. In the present embodiment, a wireless tag reader (a read circuit) that communicates with the wireless tag is provided in the input unit 503 of the determination apparatus 20. The wireless tag reader wirelessly communicates with the wireless tag of the CI 4 to determine the type of the CI 4. Also, in the first embodiment, the measurement apparatus 1 measures the profile of the CI 4 to determine the type and the orientation of the CI 4. In contrast, in the present embodiment, the measurement apparatus 1 can be provided with a wireless tag reader (a read circuit) that communicates with a wireless tag. Note that the wireless tag of the CI 4 may be located at either end of the CI 4, for example. The wireless tag reader wirelessly communicates with the wireless tag of the CI 4, and determines the type of the CI 4. Thus, the orientation of the CI 4 can be determined based on the position of the wireless tag. Due to such a process, steps S11 and S12 in FIG. 7 can be omitted.

Note that it is possible to employ a configuration in which an optical reading unit such as a barcode reader is used instead of a wireless tag. If this is the case, the identification patterns 31 are replaced with a barcode, a QR code (registered trademark), or the like, which serves as determination information. Note that, as with the case of the wireless tag, it is possible to determine the orientation of the CI 4 based on the positions of the identification patterns 31 on the CI 4. It is possible to employ a configuration in which an optical reading unit is provided in the input unit 503 of the determination apparatus 20, and thus the determination apparatus 20 determines the type of the CI 4. It is also possible to employ a configuration in which the spectrometer 200 of the measurement apparatus 1 or the optical reading unit that is separately provided reads the identification patterns 31, and thus the measurement apparatus 1 determines the type and the orientation of the CI 4. For example, the identification patterns 31 are provided near either end of the CI 4 in the conveyance direction thereof. Upon the leading end of the CI 4 in the conveyance direction thereof entering the reading area of the optical reading unit, the measurement apparatus 1 stops conveying the CI 4 and reads the identification patterns. At this time, if the identification patterns of the CI 4 are not detected, the measurement apparatus 1 conveys the CI 4 to a position at which the trailing end of the CI 4 in the conveyance direction enters the reading area of the optical reading unit, and thereafter stops the conveyance of the CI 4 and reads the identification patterns. Thus, it is possible to determine the type and the orientation of the CI 4.

As described above, according to the present embodiment, it is also possible to determine the type of the CI 4 using the wireless tag or the identification patterns 31, and even if a plurality of types of CIs 4 are used, it is possible to improve workability when determining the achievement degree of the sterilization process.

### OTHER EMBODIMENTS

Embodiments of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-116632, filed on Jun. 24, 2019, which is hereby incorporated by reference herein in its entirety.

1. A determination system comprising:
a type determination unit configured to acquire determination information for determining a type of an indicator that has a discoloration area, a color of which changes according to an achievement degree of a sterilization process, and to determine the type of the indicator;
a measurement unit configured to measure color information regarding the discoloration area; and
an achievement degree determination unit configured to determine the achievement degree of the sterilization process based on the color information regarding the discoloration area measured by the measurement unit, according to a determination criterion corresponding to the type of the indicator determined by the type determination unit.

2. The determination system according to claim 1, wherein the type determination unit acquires the determination information by optically reading an identification pattern that is provided on the indicator and indicates the type of the indicator, or by communicating with a wireless tag that is provided on the indicator and stores the determination information.

3. The determination system according to claim 2, wherein the type determination unit determines a position of the discoloration area of the indicator based on the determined type of the indicator and a position of the identification pattern on the indicator, or a position of the wireless tag on the indicator, and
wherein the measurement unit measures the color information regarding the discoloration area based on the position of the discoloration area of the indicator determined by the type determination unit.

4. The determination system according to claim 1, wherein the determination information is color information regarding a surface of the indicator in a predetermined range measured by the measurement unit.

5. The determination system according to claim 4, wherein the predetermined range is a range that includes an identification pattern that is provided on the indicator and indicates the type of the indicator.

6. The determination system according to claim 4, wherein the type determination unit determines a position of the discoloration area of the indicator based on the determined type of the indicator and the color information regarding the predetermined range of the indicator measured by the measurement unit.

7. The determination system according to claim 6, wherein the measurement unit measures the color information regarding the discoloration area based on the position of the discoloration area of the indicator determined by the type determination unit.

8. The determination system according to claim 6, wherein the predetermined range is a range that includes a portion of the discoloration area, and
wherein the achievement degree determination unit (i) determines color information corresponding to the discoloration area from the color information regarding the predetermined range based on the position of the discoloration area of the indicator determined by the type determination unit and (ii) determines the achievement degree of the sterilization process based on the color information corresponding to the discoloration area.

9. The determination system according to claim 1, wherein the measurement unit measures the color information while a relative positional relationship between the measurement unit and the indicator is changing.

10. The determination system according to claim 1, wherein the measurement unit and the type determination unit are provided in a measurement apparatus, and
wherein the achievement degree determination unit is provided in a determination apparatus that is configured to communicate with the measurement apparatus.

11. The determination system according to claim 1, wherein the measurement unit is provided in a measurement apparatus, and the type determination unit and the achievement degree determination unit are provided in a determination apparatus that is configured to communicate with the measurement apparatus.

12. A determination apparatus comprising:
a type determination unit configured to acquire determination information for determining a type of an indicator that has a discoloration area, a color of which changes according to an achievement degree of a sterilization process, and to determine the type of the indicator;
a reception unit configured to receive color information regarding a surface of the indicator in a predetermined range that includes at least a portion of the discoloration area; and
an achievement degree determination unit configured to determine the achievement degree of the sterilization process based on the color information regarding the discoloration area received by the reception unit, according to a determination criterion corresponding to the type of the indicator determined by the type determination unit.

13. The determination apparatus according to claim 12, wherein the type determination unit acquires the determination information by optically reading an identification pattern that is provided on the indicator and indicates the type of the indicator, or by communicating with a wireless tag that is provided on the indicator and stores the determination information.

14. The determination apparatus according to claim 12, wherein the determination information is color information regarding a surface of the indicator in the predetermined range received by the reception unit.

15. The determination apparatus according to claim 14, wherein the predetermined range is a range that includes an identification pattern that is provided on the indicator and indicates the type of the indicator.

16. The determination apparatus according to claim 14, wherein the type determination unit (i) determines color information corresponding to the discoloration area from the color information regarding the predetermined range based on the determined type of the indicator and the color information regarding the predetermined range of the indicator received by the reception unit and (ii) determines the achievement degree of the sterilization process based on the color information corresponding to the discoloration area.

17. A measurement apparatus comprising:
a position determination unit configured to acquire determination information for determining a position of a discoloration area, a color of which changes according to an achievement degree of a sterilization process, of an indicator, and determining the position of the discoloration area of the indicator;
a measurement unit configured to measure color information regarding the discoloration area based on the position of the discoloration area determined by the position determination unit; and
an output unit configured to output the color information regarding the discoloration area measured by the measurement unit.

18. A measurement apparatus comprising:
a position determination unit configured to acquire determination information for determining a position of a discoloration area, a color of which changes according to an achievement degree of a sterilization process, of an indicator, and determining the position of the discoloration area of the indicator;
a measurement unit configured to measure color information regarding a surface of the indicator in a predetermined range that includes at least a portion of the discoloration area; and
an output unit configured to determine color information corresponding to the discoloration area, from the color information regarding the predetermined range of the indicator measured by the measurement unit, based on the position of the discoloration area determined by the position determination unit, and to output the color information corresponding to the discoloration area.

19. The measurement apparatus according to claim 17, wherein the determination information is information that is provided on the indicator and indicates a type of the indicator, and
wherein the position determination unit determines the position of the discoloration area of the indicator based on the type of the indicator and the position on the indicator at which the information indicating the type of the indicator is provided.

20. The measurement apparatus according to claim 19, wherein the information indicating the type of the indicator is an identification pattern that is provided on the indicator, or information that is stored in a wireless tag that is provided on the indicator.

21. The measurement apparatus according to claim 17, wherein the determination information is color information measured by the measurement unit regarding a surface of the indicator in a predetermined range that includes at least a portion of the discoloration area, and
wherein the position determination unit determines the type of the indicator based on the color information regarding the predetermined range, and determines the position of the discoloration area of the indicator based on the determined type of the indicator and the color information regarding the predetermined range.

22. The measurement apparatus according to claim 21, wherein the predetermined range is a range that includes an identification pattern that is provided on the indicator and indicates the type of the indicator.

23. The measurement apparatus according to claim 17, wherein the measurement unit measures the color information while a relative positional relationship between the measurement unit and the indicator is changing.

24. A non-transitory computer-readable storage medium that stores a program, wherein, upon being executed by one or more processors of one or more apparatuses, the program causes the one or more apparatuses to:
acquire determination information for determining a type of an indicator that has a discoloration area, a color of which changes according to an achievement degree of a sterilization process;
determine the type of the indicator;
measure color information regarding the discoloration area; and
determine the achievement degree of the sterilization process based on the measured color information regarding the discoloration area, according to a determination criterion corresponding to the determined type of the indicator.

25. A non-transitory computer-readable storage medium that stores a program, wherein, upon being executed by one or more processors of an apparatus, the program causes the apparatus to:
acquire determination information for determining a type of an indicator that has a discoloration area, a color of which changes according to an achievement degree of a sterilization process;
determine the type of the indicator;
receive color information regarding a surface of the indicator in a predetermined range that includes at least a portion of the discoloration area;
determine the achievement degree of the sterilization process based on the received color information regarding the discoloration area, according to a determination criterion corresponding to the determined type of the indicator.
